(19) United States
(12) Patent Application Publication
(10) Pub. No.: US 2006/0110375 A1
(43) Pub. Date: May 25, 2006

(54) Method for producing cell lines and organs by means of differentiable cells

(76) Inventors: Herbert Zech, Bregenz (AT); Nicolas Zech, Bregenz (AT); Gottfried Dohr, Graz (AT); Pierre Vanderzwalmen, Lasne (BE)

Correspondence Address: WILLIAM COLLARD;COLLARD & ROE, P.C., 1077 NORTHERN BOULEVARD, ROSLYN, NY, 11576, US

(21) Appl. No.: 10/524,187

(22) PCT Filed: Aug. 11, 2003
(86) PCT No.: PCT/AT03/00232

§ 371 Date: Sep. 30, 2005

(30) Foreign Application Priority Data
Aug. 9, 2002 (AT) A1206/2002

(51) Int. Cl. A61K 48/00; C12N 5/06

(52) U.S. Cl. 424/93.21; 435/325; 435/354

(57) ABSTRACT

A method for producing cell lines or individual organs, differentiable donor cells being supplied to a morula or blastocyst which is cultivated under conditions that ensure a further development of the morula or blastocyst in stages in which newly formed cell lines having a higher degree of differentiation occur, as well as isolation of the cell lines or further differentiation of the cell lines into organs. The method according to the present invention is distinguished in that the cells of the morula or the internal cell mass of the blastocyst have a restricted survivability in comparison to the particular wild type or their survivability is reduced through suitable cultivation conditions, and the donor cells supplied to the morula or blastocyst have varying degrees of differentiation. A significantly shortened cultivation period of the donor cells may thus be achieved in comparison to typical methods, through which the provision of natural stem cells is favored, and perhaps made possible for the first time. The blastocysts may also be used by being transferred into a surrogate mother animal.

The present invention relates to a method for producing cell lines and organs with the aid of differentiable cells according to the preamble of claim 1.

Pluripotent cells, as occur in early stages of embryonic development, as well as cells of the germ line, are a special type of differentiable cells. In the following, pluripotent cells are understood as cells which may differentiate into every cell type. A property comparable to pluripotence, but probably based on plasticity, may, however, also be induced in cells of later development stages through technical measures, such as those according to the method according to the present invention. These cells are not included in the following by the term “pluripotent cells”. Pluripotent cells have the capability of producing all cell types of the embryo, the fetus, and the adult organism, as well as regenerating themselves nearly infinitely. A renewable source of cells which may be differentiated into manifold different types of tissue certainly offers manifold application possibilities in basic research and in transplant therapy. An important step for implementing this goal is represented by the discovery that human, embryonic stem cells may be cultivated [3]. In the following, “embryonic stem cells” are understood as pluripotent cells which are removed from a morula or blastocyst and preferably kept viable in culture dishes. Obtaining and cultivating them is well known to those skilled in the art and was disclosed, for example, in U.S. Pat. No. 6,011,197 and WO 97/37009. These embryonic stem cells (ES cells) are primarily obtained in this case from the internal cell mass of blastocysts, i.e., those cells of the blastocysts from which all cells of the later endoderm, ectoderm, and mesoderm finally arise. It is noted at this point that in the following a “pre-embryo” is understood as a developing cell mass up to day 6 after fertilization of the egg cell, and after day 6, and therefore possibly after implantation in the birth mother, reference is made to an “embryo”. In particular, the term “pre-embryo” here comprises the preimplantation stages from the zygote via the morula up to the blastocyst until day 6 after fertilization of the egg cell. The embryo in the single-cell stage (pronucleus stage) is identified here as a zygote. Preimplantation stages may be initiated through fertilization of an egg cell by a sperm, through parthenogenetic activation of an egg cell, or through addition of one or more blastomers into an inductive environment such as a zona pellucida, as was described by Alikani and Willadsen [11]. The term morula refers here to all stages of cell divisions following the zygote, including the early cell division stages on days 2 and 3, in which a blastocoel has not yet formed. After formation of a blastocoel, reference is made in the following to a blastocyst, this term also able to refer to early embryonic stages. The blastocysts are formed by the zona pellucida (external, non-cellular mass) and by the trophoblasts and contain the internal cell mass already cited. After the hatching from the zona pellucida around day 6, this structure is also referred to as the blastocyst and is an embryo according to the above terminology. Methods for isolating an internal cell mass from a blastocyst are known to those skilled in the art [8, 9]. “Embryonic stem cells” or “ES cells” would therefore actually be “free embryonic stem cells” according to this terminology, if they were obtained from the morula or the blastocyst up to day 6, but the term “embryonic stem cells” or “ES cells” is generally maintained for pluripotent stem cells which were obtained from the morula or blastocyst, even if the removal is to be performed before day 6.

The potential of pluripotent cells such as ES cells for research and clinical use is extensive. Their future significance for in vitro studies of human embryogenesis, for investigations of abnormal development (for example, through the production of cell lines having intentional gene alterations), for the investigation of the effect of individual genes, for the development and testing of novel medications, or as a renewable source for cell and tissue transplants or for genetic therapies may currently hardly be estimated.

A further impetus for research of this type is provided by the observation that ES cells of a first species may be introduced into the blastocyst of a second species, even a different one, which, after transfer into a female of the second species, leads to the birth of an offspring that combines genetic features of both species and thus represents a chimera, which is also understood in the following as a developing cell mass that contains a subgroup of cells which have DNA having significantly different nucleotide base sequences in the cell nuclei than the other cells of the cell mass.

In order to elevate the genetic contribution of the donor ES cells to the finally resulting organism, it was suggested in US 2002062493, for example, that non-human mammals be produced by injecting ES cells of the relevant animal into tetraploid blastocysts of the same species. The blastocysts are cultivated until development of an embryo and transferred to a female to be carried until delivery. In a further embodiment, the ES cells are provided with mutations and then injected into tetraploid blastocysts, through which offspring having intentional mutations may be generated. Methods of this type have been ascribed with a large potential for the research of the effect of individual genes and/or their mutations on the phenotypic development of offspring.

To produce tetraploid blastocysts, typically the blastomers of diploid pre-embryos in the two-cell stage are fused by applying brief electrical pulses. The embryos thus arising may be cultivated and result in the development of morulae and blastocysts, the tetraploidy manifesting in the cells of the internal cell mass, for example, in the latter.

Morulae may be used for aggregation with ES cells and blastocysts for injection of ES cells, for example. However, tetraploid blastocysts are distinguished by a restricted development capability. While the differentiation of tetraploid cells hardly exceeds the development of early endoderm and trophoectoderm, injected diploid ES cells may result in the development of a mature embryo, for example. Therefore, these methods were suggested, as described in US2002062493, for example, in order to produce chimeras effectively, since due to the reduced lifespan of the tetraploid cells of the internal cell mass, the unfolding of the phenotype of the host is suppressed naturally in favor of the ES cells of the donor organism. In particular, it was suggested that the phenotypic contribution of specific genes be determined rapidly with the aid of genetically modified ES cells [1].

In this context, it is to be noted that all attempts to induce a normal fetal development by completely removing the internal cell mass and replacing it by ES cells have been shown to be failures, although ES cells have been ascribed with the capability of producing the entire fetus. Therefore, it is assumed that the internal cell mass of the host apparently exerts a decisive function of inducing the ES cells to reenter into an embryonic differentiation program, even if the cells of the internal cell mass are tetraploid cells having restricted viability.

Obtaining, using, and genetically altering ES cells encounters ethical considerations, particularly with human ES cells, so that alternatives are to be sought, both in regard to the use of human blastocysts and also human ES cells in research and clinical therapy.

The use of embryonic stem cells also encounters technical difficulties, however. These cells may thus currently only be obtained from pre-embryos or very early embryos and are not immunologically compatible with most patients, although multiple ES cell lines have currently been isolated. A possible explanation may be that human ES cells express MHC-I [10]. Therefore, it will either be necessary to isolate multiple further ES cell lines or tailor ES cell lines to each patient with the aid of “therapeutic cloning”. Furthermore, ES cells tend to form teratomas after being transplanted. ES cells must therefore be differentiated reliably into appropriate tissue types during their cultivation, before being transplanted. In addition, the question arises of whether specialized cells, which were derived from ES cells, also have the desired functional properties in corresponding tissue after being transplanted. Thus, for example, it has been shown that mouse ES cells which produced insulin in vitro could not cause any reduction of the blood sugar level in vivo.

Therefore, alternatives to the use of ES cells have entered the focus of research interest. Thus, the question arises of whether adult stem cells, possibly even from umbilical cord blood, may also be used as a replacement for ES cells, since it appears obtaining them from samples of adult organisms, for example, seems less ethically questionable, even in humans, than obtaining ES cells from pre-embryos or early embryos. This initially appeared impossible because of the restricted differentiability of adult stem cells.

As the most recent investigations have shown, however, MAPCs (“multipotent adult progenitor cells”) may be obtained upon purifying mesenchymal stem cells of mice, which differentiate not only into mesenchymal cells, but rather also into cells of the endoderm, mesoderm, or ectoderm [2]. If MAPCs are injected into early mouse blastocysts, for example, it may be determined that they contribute to the formation of multiple, possibly even all somatic cell types. These investigations are interesting in that it had been assumed until now that tissue-specific stem cells, which surely have less capability for self-regeneration available than ES, generally differentiate only into cells of the relevant tissue. It has been observed that hematopoietic stem cells may also differentiate into cells of other tissue types under certain circumstances or that neuronal stem cells may contribute to multiple tissue types after injection into a blastocyst, but it was typically thought to be practically impossible for a single tissue-specific stem cell to be able to differentiate into functional cells of multiple tissue types. As the investigations have also been able to show, MAPCs also contribute in vivo to the formation of multiple somatic tissue types when they are administered to a mouse. In fact, it has been shown that MAPCs are similar to ES cells in their properties and, in addition, represent a very synchronous cell type in regard to their differentiability, as has been shown on the basis of investigations of gene expression. They require approximately comparable culture conditions to ES cells, express at least some of the genetic markers which are also observed in ES cells in vitro (Oct-4, Rex-1, SSEA-1), but have pronounced proliferation and differentiation properties, possibly contribute to the formation of all organs when they are injected into blastocysts, and differentiate into tissue-specific cells when they are subjected to the corresponding influence of the relevant organs.

The nature of these MAPCs is currently still unexplained, and it even appears questionable whether the MAPCs observed in vitro, which are the result of comparatively long cultivation periods of multiple months, also exist in this form in vivo. Thus, it has been speculated that MAPCs actually do not exist in vivo, but rather that cells having altered properties, in some circumstances also similar to those of cancer cells, were cultivated. According to a further explanation model, the long cultivation period may encourage the reduction of the original cell population to included stem cells, as was observed in the hematopoietic cells, which are very similar in their properties.

Finally, it was disclosed in EP 1176189 that cells may be obtained from samples of adult somatic cells, from muscle tissue, brain tissue, the blood, the bone marrow, the liver, or the mammary glands, for example, which display behavior similar to the pluripotent stem cells, and particularly display expression of Oct-4, as is also the case in pluripotent stem cells in early stages of embryonic development. These cells were also referred to as “de-differentiated” stem cells, in order to thus express the suspicion that cells may apparently regain greater differentiability. However, more precise statements about their usability for the production of differentiated or novel, differentiable cells were not provided.

It is the object of the present invention to provide a method for producing differentiated or novel, differentiable cell lines or even entire organs, without using differentiable cells that were obtained from pre-embryos or embryos, such as ES cells. It is a further object of the present invention to achieve this while avoiding long cultivation periods of the differentiable cells used. Notwithstanding the risks already noted of long cultivation periods in connection with the above-mentioned MAPCs, hazards are also outlined in this regard on the basis of “genetic imprinting” [4, 5].

The object of the present invention is achieved by the characterizing features of claim 1.

In claim 1, to produce differentiated or novel, differentiable cell lines or even organs, embryonic stem cells or stem cell lines which are to have a uniform degree of differentiation in regard to their pluripotence, are not used, as is described in U.S. Pat. No. 6,200,806 or in [12], but rather cells having a primary varying degree of differentiation, which particularly characterizes a sample of a donor organism containing adult, somatic stem cells. In this case, “varying degree of differentiation” of donor cells is understood to mean that they may comprise multipotent/pluripotent or even differentiated cells, a relatively large number of differentiated cells or cells which can hardly be differentiated further typically being found in a sample of adult cells of a donor organism and only a small number of cells still having multipotent/pluripotent character. The donor cells are certainly cell populations which have experienced preparation with the aid of suitable methods to increase the concentration of included stem cells, for example, in the course of the production of a highly-purified fraction from umbilical cord blood, but obtaining synchronous cell populations, i.e., cells having a uniform degree of differentiation, as is the case when obtaining embryonic stem cell lines, and the long cultivation period connected therewith, is dispensed with.

Even if it was conceivable to synchronize the sample in regard to the differentiability of the included cells with the aid of sufficiently long cultivation periods, this is not necessary if it is introduced according to the present invention into morulae or blastocysts, whose cells, in blastocysts those of the internal cell mass, have a survivability restricted in comparison to the wild type morula and/or wild type blastocyst, or if the survivability of these cells is reduced through suitable cultivation conditions.

In the following, a wild type morula and/or wild type blastocyst is understood as a morula or blastocyst which has not yet experienced any manipulation in this connection. As was already noted, the internal cell mass of the host blastocyst apparently exerts a decisive function in regard to inducing the ES cells introduced into the blastocyst to reenter an embryonic differentiation program, even if the cells of the internal cell mass are cells having restricted survivability, because of tetraploidy, for example. As has now surprisingly been determined, it is possible through of the “reprogramming” cell matrix provided by the cells of the internal cell mass, but also the cells of the morula, to cause a “de-differentiation” in regard to greater differentiability even in non-embryonic stem cells. It is also conceivable that it is more appropriate to speak of a “trans-differentiation” of the supplied donor cells, their plasticity being caused to unfold by being embedded in an appropriately stimulating cellular environment. The mode of operation of the “stimulation” is currently not completely explained, but both intercellular factors, such as autocrine and paracrine factors, and also the “polarity” of the embryo appear to play a role, in addition to the extracellular matrix. The present invention is therefore based on the idea that possible deficits in regard to the differentiability of stem cells which were not obtained from pre-embryos or early embryos may be compensated for through their contact with an appropriately reprogramming cell matrix, consideration having to be taken that, in regard to an optimized yield of newly-formed cell lines, the cells of the internal cell mass of the blastocyst and/or the cells of the morula must have a restricted survivability in comparison to the wild type blastocyst and/or wild type morula, or their survivability must be reduced through suitable cultivation conditions. The cells of the internal cell mass and/or the cells of the morula therefore support the desired reprogramming of the donor stem cells supplied, although their proportion in the cells of the developing organism is continuously reduced because of their restricted survivability.

“Restricted survivability” of the cells of the morula and/or internal cell mass of the blastocyst may be produced in different ways. Either the restricted survivability is already provided “intrinsically”, as in tetraploid embryos, or restricted survivability of certain cells may be induced “extrinsically” with the aid of suitable cultivation conditions. Both possibilities will be discussed in the following.

Claim 2 provides a preferred embodiment, according to which the donor cells contain naturally occurring stem cells. “Naturally occurring” stem cells are understood here as adult, somatic stem cells, also from umbilical cord blood, as may be found in vivo. In regard to the above-mentioned MAPCs, it was determined that MAPCs isolated after long cultivation periods of multiple months may not actually exist in vivo, but rather cells having altered properties were cultivated because of the long cultivation period. It is also not entirely to be excluded in the method according to the present invention that, in spite of the significantly shorter preparation and cultivation periods, the donor cells represent a cell population altered in comparison to the natural stem cells occurring in vivo. In the preferred embodiment of the method according to the present invention, the donor cells contain naturally occurring stem cells, however, which is also favored by a rapid preparation and cultivation of the donor cells, in addition to the use according to the present invention of donor cells having varying degrees of differentiation.

Claim 3 provides a special embodiment of the method according to the present invention, according to which the cells of the morula or the internal cell mass of the blastocyst are prepared as a reaction medium in a culture dish. However, it would also be conceivable to use the cells of the morula or the internal cell mass of the blastocyst to prepare a reaction medium as described in [37], since it is currently assumed that dissolved matrix components such as laminin, collagen IV, cytokine, or even proteins existing on the cells, such as glycoproteins, may also act as a “reprogramming” cell matrix. A standard medium is preferably used for this purpose, which preferably dispenses with the use of FCS (“fetal calf serum”) or another serum derived from animal proteins.

According to claim 4, the donor cells are obtained from umbilical cord blood, particularly by producing a highly purified fraction which contains approximately 5% stem cells.

According to claim 5, the donor cells are obtained from the placenta. The placenta contains multiple cells which are of interest for the method according to the present invention, such as mesenchymal cells and endothelial cells, which are assumed to be an especially bountiful source for stem cells. According to claim 6, the donor cells are obtained from the bone marrow. According to claim 7, the donor cells are obtained from the fatty tissue, which is distinguished as an especially interesting source, since stem cells from the fatty tissue are relatively easy to obtain. Containing the donor cells comprises preparing the sample, taken from an adult organism, the preparation having the goal of elevating the concentration of stem cells contained in the sample. Methods of this type are related art and are well known to those skilled in the art.

According to claim 8, the cells of the receiver morula and/or the internal cell mass of the receiver blastocyst are tetraploid cells. Methods for implementing such a tetraploidy are known according to the related art [6, 7]. As has already been noted, tetraploid cells have a restricted survivability. Because of the restricted survivability intrinsically provided in the tetraploid cells of the morula and/or the internal cell mass of the blastocyst, their number is gradually reduced and an increasing popularization of the developing blastocyst with successor cells of the donor cells supplied is thus ensured, the tetraploid cells setting the required intercellular signals for reprogramming the donor cells supplied in regard to greater differentiability over the duration of their existence.

Claim 9 provides an alternative method for equipping the cells of the morula or the internal cell mass of the blastocyst with a restricted survivability, in that a vector is incorporated into their genome which causes a lethal sensitivity to appropriately selected cultivation conditions. If the cultivation conditions are selected accordingly after the donor cells are supplied to the morula and/or the blastocyst, this results in intentional dying of the cells of the morula and/or the internal cell mass, without impairing the survivability of the donor cells and the trophoblasts. Thus, for example, vectors may be selected which cause a higher sensitivity to temperature increase or specific additives for culture media.

As an alternative to this, according to claim 10, the genome of the donor cells is provided with a vector such as a neomycin-resistance gene or puromycin-resistance gene, which causes a resistance to media having additives such as G 418 or puromycin. However, it must be ensured for this purpose that the incorporation of the vector into the donor cells does not cause cultivation times that impair the object according to the present invention of the shortest possible cultivation times of the donor cells before they are supplied to the blastocyst. Vectors may also be supplied which make the cells resistant to specific temperature influences, such as temperature increases [13-17].

As a further alternative, according to claim 11 the survivability of the cells of the morula and/or the internal cell mass of the blastocyst is reduced by adding suitable antibodies to the culture media. By adding specific antibodies (AB), which are charged with cell-damaging substances and only adhere to cells that have a receptor for the specific AB, only these cells are thus damaged. Techniques of this type are described, for example, in [18-22].

Implementing claims 9 through 11 allows an advantageous embodiment of the method according to the present invention according to claim 12 to be implemented, according to which the reduction of the survivability of the cells of the morula and/or the internal cell mass of the blastocyst is performed in a way which is tailored to the varying degrees of differentiation of the donor cells and is chronologically well-ordered. A varying composition of the donor cells introduced into the host morula and/or host blastocyst may specifically require the cells of the morula and/or the internal cell mass of the blastocyst to die in a chronologically tailored way, in order to achieve an optimized signal setting of the reprogrammed cell matrix. The intentional reduction of the survivability of the cells in the morula and/or the internal cell mass must ensure, however, that the trophoblasts are not impaired in their survivability, since they are important for the further survival of the embryo, particularly if the blastocysts are transferred into a surrogate mother animal.

The cell sample obtained from a cell sample of the donor organism or from umbilical cord blood represents, as noted, even after purification of the sample in regard to the differentiability of the included cells, an asynchronous cell population, which not only contains multipotent, at best even pluripotent stem cells, but rather also cells having lesser differentiability, such as tissue-specific cells, which may complete a trans-differentiation into tissue-specific cells of other tissues under specific circumstances, however, or even differentiated cells possibly without any differentiability. Therefore, it may be advantageous to bring the donor cells into contact with other blastocysts or isolated internal cell masses of other blastocysts in culture dishes according to claim 13 before the donor cells are supplied to the morula and/or blastocyst. Methods for isolating and cultivating internal cell masses are known to those skilled in the art, a medium of undifferentiated cells finally being prepared from isolated internal cell masses, onto which the donor cells may be applied or washed with a high probability of contact with the cells of the prepared internal cell mass. Specifically, it has been shown that through the contact with blastocysts or isolated internal cell masses of blastocysts, a selection of cells may be achieved which is suitable for the method according to the present invention in regard to higher differentiability. Donor cells having a relatively high affinity to the medium prepared from internal cell masses or the blastocysts may be isolated and are available for further therapeutic, diagnostic, or scientific applications, but may also be injected into morulae or blastocysts for further differentiation. In the latter case, the probability of induction of a higher differentiability of the injected donor cells by the cell matrix of the morula and/or the internal cell mass of the host blastocysts is elevated, the duration of this additional method step only being a few minutes, or even a few seconds if the donor cells are washed onto the medium prepared from internal cell masses, so that even if this step is implemented, the cultivation periods may be kept comparatively short.

As an alternative to this, the features of claim 14 may also be implemented, according to which cells suitable for the method according to the present invention are preselected via appropriate markers.

Claims 15 and 16 provide special embodiments of the method according to the present invention. Particularly if human donor cells are used and are injected into pig blastocysts, for example, the possibility arises via the method according to the present invention of producing cell lines whose genetic properties are comparable, and in the best case identical, to those of the original, human donor cells, although the pig blastocyst developing after application of the method according to the present invention is in no way genetically identical to the donor cells. In a later development stage of the pig blastocyst, novel, differentiable cells, differentiated cell lines, or even entire organs having genetic identity to the human donor cells and in the optimum case also immunological compatibility with the donor organism may be isolated, without this requiring the use of human embryonic stem cells.

Claim 17 provides an advantageous type of the supply of the donor cells into the host blastocyst, in that the supply is performed through injection.

Claim 18 provides an advantageous type of the supply of the donor cells into the host morula, in that the supply is performed through aggregation.

Claim 19 relates to a special embodiment of the method according to the present invention, according to which the donor cells are human cells. However, it is thoroughly possible for the morula or blastocysts to which the donor cells are supplied to nonetheless be of non-human origin. Since the cell lines or even organ structures harvested from the method according to the present invention are genetically identical to the donor cells, they are suitable for use as a preparation for therapeutical intervention according to claims 20 and 21, for example, for illnesses as are cited in claim 22.

Claim 23 relates to a special embodiment of the method according to the present invention, according to which the donor cells are non-human cells. Since the cell lines harvested from the method according to the present invention are again genetically identical to the donor cells, they are suitable for use as a preparation for therapeutic and diagnostic intervention in the veterinary field according to claim 24, and for producing genetically identical cells and organ structures for therapeutic, diagnostic, or scientific application according to claim 25, for illnesses as are cited in claim 26, for example.

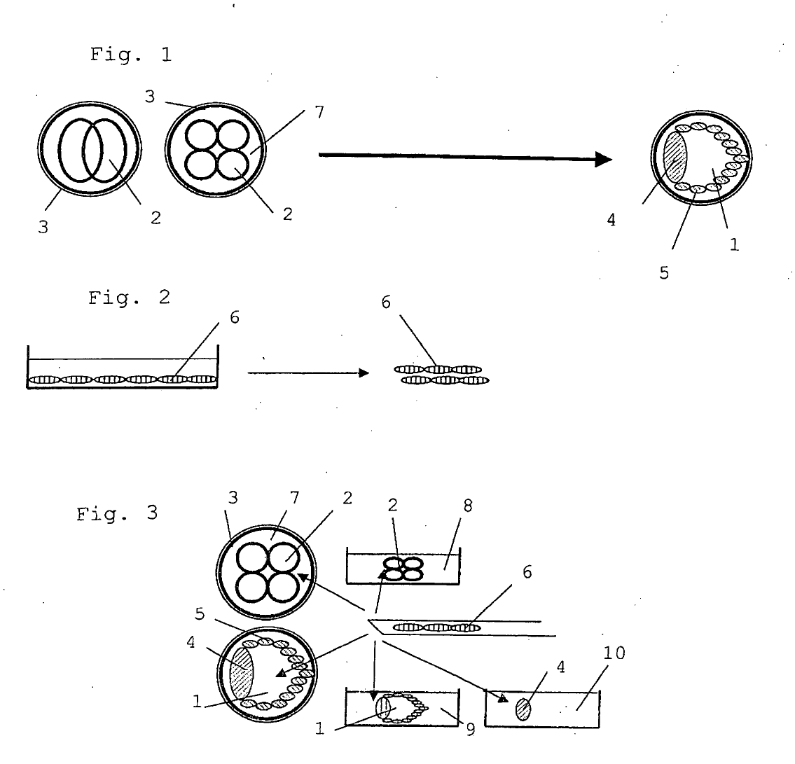

A possible embodiment of the method according to the present invention will now be described in greater detail on the basis of the attached FIGS. 1 through 3.

For this purpose, FIG. 1 is to schematically illustrate how, according to an embodiment of the method according to the present invention, a tetraploid blastocyst 1 is first produced. The blastomers 2, surrounded by the zona pellucida 3, of a two-cell pre-embryo may be converted through electrofusion, for example, into a one-cell pre-embryo having a tetraploid chromosome number. Techniques for producing tetraploid pre-embryos are known from the related art and described, for example, in [6, 7, 23-27].

Furthermore, the pre-embryo completes cell divisions of the blastomers and develops further into a blastocyst 1. The internal cell mass 4 and the trophoblasts 5 are indicated schematically in FIG. 1.

In addition, a sample originating from umbilical cord blood, for example, or even a sample taken from an adult organism, from fatty tissue, for example, is subjected to a preparation which is intended to elevate the concentration of the included stem cells. Techniques for preparing a sample for the purpose of producing a purified cell fraction are also known according to the related art and described, for example, in [28, 32, 34]. The result of the preparation is donor cells 6 (FIG. 2), which have varying degrees of differentiation and may comprise multipotent/pluripotent or even differentiated cells, a relatively large number of differentiated or hardly still differentiable cells being found in a sample of adult cells of the donor organism and only a small number of cells still having multipotent/pluripotent character. The differentiation potential is known for some types of adult stem cells according to the related art [e.g., 29, 30, 31, 34].

In the framework of the method of according to the present invention, the donor cells 6 are not synchronized in regard to the differentiability of the included cells with the aid of sufficiently long cultivation periods, but rather they are introduced into morulae 7 or blastocysts 1, whose cells 2, in blastocysts those of the internal cell mass 4, now have restricted survivability, because of the tetraploidy produced, in comparison to the wild type morula and/or wild type blastocyst (FIG. 3). As was already noted, the cells 2 of the host morula 7 and/or the internal cell mass 4 of the host blastocyst 1 apparently exert a decisive function in inducing introduced stem cells to reenter an embryonic differentiation program. As has now surprisingly been determined, it is possible because of the “reprogramming” cell matrix provided by the cells of the internal cell mass 4, but also by the cells 2 of the morula 7, to cause a “de-differentiation” in regard to greater differentiability, even in non-embryonic stem cells. As was already noted, it may be more relevant to speak of a “trans-differentiation” of the supplied donor stem cells 6, their plasticity being caused to unfold by embedding them in an appropriately stimulating cellular environment. The present invention is therefore based on the idea that possible deficits in regard to the differentiability of stem cells which were not obtained from pre-embryos or early embryos may be compensated for through their contact with an appropriately reprogramming cell matrix, care having to be taken that, in consideration of an optimized yield of newly-formed cell lines, the cells of the internal cell mass 4 of the blastocyst 1 and/or the cells 2 of the morula 7 have a restricted survivability in comparison to the wild type blastocyst and/or wild type morula. The cells of the internal cell mass 4 and/or the cells 2 of the morula 7 thus do support the desired reprogramming of the supply donor stem cells 6, although their proportion in the cells of the developing organism continuously decreases because of their restricted survivability. As is also indicated in FIG. 3, the cells 2 of the morula 7 may be prepared in a culture dish 8 in this case. As an alternative to this, the cells of the internal cell mass 4 of the blastocyst 1 may also be prepared in a culture dish 10. Furthermore, it is also conceivable to perform the cocultivation in a culture dish 9 with the aid of appropriately prepared blastocysts 1. Techniques for the cocultivation of stem cells with other cell types are described, for example, in [35, 36].

Particularly if human donor cells 6 are used and are injected, for example, into pig blastocysts 1, the possibility arises via the method according to the present invention of producing cell lines whose genetic properties are comparable, and in the best case identical, to those of the original, human donor cells 6, although the pig blastocyst 1 developing after application of the method according to the present invention is in no way genetically identical to the donor cells 6. In a later development stage of the pig blastocyst 1, novel, differentiable cells or differentiated cell lines having genetic similarity and/or identity to the human donor cells 6 and in the optimum case also immunological compatibility to the donor organism may be isolated, without this requiring the use of human embryonic stem cells. Preparations for multiple human illnesses, as listed in the claims, may be manufactured from these cell lines.

Upon transfer of the pig blastocyst 1 into a surrogate mother animal, it is also conceivable to allow the development of organs with genetic similarity and/or identity to the human donor cells 6 and in the optimum case also immunological compatibility to the donor organism. Thus, with the aid of the method according to the present invention, for example, a heart of a pig made of up to 100% human cells may result from a human donor cells 6 which were obtained without the use of a pre-embryo or embryo, using a pig blastocyst, without causing suffering of the affected animal in this case. In the optimum case, the heart would be available with complete immunological compatibility to the donor organism.

### LITERATURE

[1] Wang et al., Mechanisms of Development 62 (1997), S. 137-145
[2] Jiang et al., Nature 418, 41-49 (2002)
[3] Thomson et al., Science 282, 1145 (1998)
[4] Li et al., Nature 366, 362-365 (1993)
[5] Li et al., Cell 69, 915-926 (1992)
[6] James et al., Genet. Res. Camb. 60, 185 (1992)
[7] Wang et al., Mech. Dev. 62, 137 (1997)
[8] Sims & First, Proc. Natl. Acad. Sci. USA 91, 6143 (1994)
[9] Keefer et al., Mol. Reprod. Dev. 38, 264 (1994)
[10] Drukker et al., PNAS Vol. 99 No. 15, 9864 (2002)
[11] Mina Alikani, Steen M Willadsen. Reproductive BioMedicine Online 2002 Vol. 5, No. 1 56-58
[12] Smith, Annual Review, 17 S. 449, 450 (2001)
[13] Heads R J, Latchman D S, Yellon D M. J Mol Cell Cardiol 1994 June;26(6):695-9
[14] Conneally E, Bardy P, Eaves C J, Thomas T, Chappel S, Shpall E J, Humphries R K. Blood 1996 Jan. 15;87(2):456-64
[15] Mehlen P, Preville X, Chareyron P, Briolay J, Klemenz R, Arrigo A P. J Immunol 1995 Jan. 1;154(1):363-74
[16] Sass H. Gene 1990 May 14;89(2):179-86
[17] Watanabe S, Kai N, Yasuda M, Kohmura N, Sanbo M, Mishina M, Yagi T. Biochem Biophys Res Commun 1995 Aug. 4;213(1):130-7
[18] Tietze L. F. et al. Angewandte Chemie International Edition, 41, 759-761, (2002)
[19] McDevitt M R et al. Science 2001 Nov. 16;294(5546):1537-40
[20] Tietze L F et al. Chembiochem 2001 Oct. 1;2(10):758-65
[21] Tietze L F et al. Chembiochem 2002 Mar. 1;3(2-3):219-25
[22] Tietze L F et al. Chembiochem 2001 May 4; 2(5): 326-34.
[23] Nagy et al. Development 1990;110:815-822
[24] Nagy A. et al. Proc Natl Acad Sci U S A 1993 Sep. 15;90(18):8424-8
[25] Eggan K. et al. Proc Natl Acad Sci USA 2001;98:6209-6214.
[26] Dean et al. 1998. Development 125, pp. 2273-2282.
[27] Amano T et al. Zygote 2001 May;9(2):153-7
[28] Lagasse E et al. Nat Med 2000;6:1229-1234
[29] Krau se D S et al. Cell 2001;105:369-377
[30] Galli R et al. Nat Neurosci 2000;3:986-991.
[31] Clarke D L et al. Science 2000;288:1660-1663.
[32] Conneally E et al. Proc Natl Acad Sci U S A 1997 Sep. 2;94(18):9836-41
[33] Pafumi C et al. Pediatr Hematol Oncol 2002 June;19(4):239-45
[34] Hung S C et al. Stem Cells 2002;20(3):249-58
[35] Mollah Z U, et al. J Invest Dermatol 2002 March;118(3):450-60
[36] Buzanska L et al. J Cell Sci 2002 May 15;115(Pt 10):2131-8
[37] Xu et al. Nat. Biotech. Vol. 19, Nr. 10 (2001)

1. A method for producing cell lines or individual organs, differentiable donor cells (6) being supplied to a nonhuman morula (7) or nonhuman blastocyst (1), which are cultivated under conditions that ensure a further development of the morula (7) or blastocyst (1) in stages in which newly formed cell lines having a higher degree of differentiation occur, and comprising the isolation of the cell lines or further differentiation of the cell lines into organs through transfer of the blastocyst (1) into a surrogate mother animal,
wherein the cells (2) of the morula (7) or the internal cell mass (4) of the blastocyst (1) have a restricted survivability in comparison to the particular wild type or their survivability is reduced through suitable cultivation conditions, and the donor cells (6) supplied to the morula (7) or blastocyst (1) have varying degrees of differentiation and are of non-embryonic origin.

2. The method according to claim 1,
wherein the donor cells (6) contain naturally occurring stem cells.

3. The method according to claim 1,
wherein the cells (2) of the morula (7) or the internal cell mass (4) of the blastocyst (1) are prepared in a culture dish (8, 9, 10) or are used to prepare a soluble matrix fraction.

4. The method according to claim 1,
wherein the donor cells (6) are obtained from umbilical cord blood.

5. The method according to claim 1,
wherein the donor cells (6) are obtained from placenta.

6. The method according to claim 1,
wherein the donor cells (6) are obtained from bone marrow.

7. The method according to claim 1,
wherein the donor cells (6) are obtained from fatty tissue.

8. The method according to claim 1,
wherein the cells (2) of the morula (7) or the internal cell mass (4) of the blastocyst (1) are tetraploid cells.

9. The method according to claim 17,
wherein the cells (2) of the morula (7) or the internal cell mass (4) of the blastocyst (1) has cells whose genome contains vectors that cause a lethal sensitivity to appropriate cultivation conditions in comparison to the particular wild type.

10. The method according to claim 1,
wherein the genome of the donor cells (6) contains a vector which causes a resistance to additives of culture media.

11. The method according to claim 1,
wherein the survivability of the cells (2) of the morula (7) or the internal cell mass (4) of the blastocyst (1) is reduced by adding suitable antibodies.

12. The method according to claim 9,
wherein the survivability of the cells (2) of the morula (7) or the cells of the internal cell mass (4) of the blastocyst (1) is reduced in a way that is tailored to the varying degrees of differentiation of the donor cells (6) and is chronologically well-ordered.

13. The method according to claim 1,
wherein before the donor cells (6) are supplied into the morula (7) or the blastocyst (1), the donor cells (6) are brought into contact in culture dishes with other blastocysts or internal cell masses isolated from other blastocysts, and those donor cells having a relatively high contact affinity are isolated and supplied to the morula (7) and/or blastocyst (1) first cited.

14. The method according to claim 1,
wherein before the donor cells (6) are supplied into the morula (7) or the blastocyst (1), the donor cells (6) are equipped with a genetic marker that ensures cells having a lower degree of differentiation are isolated and supplied into the morula (7) or blastocyst (1).

15. The method according to claim 1,
wherein the morula (7) or blastocyst (1) is a mouse morula or mouse blastocyst.

16. The method according to claim 1,
wherein the morula (7) or blastocyst (1) is a pig morula or pig blastocyst.

17. The method according to claim 1,
wherein when the donor cells (6) are supplied to a blastocyst (1), the supply is performed through injection.

18. The method according to claim 1,
wherein when the donor cells (6) are supplied to a morula (7), the supply is performed through aggregation.

19. The method according to claim 1,
wherein the donor cells (6) are human donor cells.

20. (canceled)

21. (canceled)

22. (canceled)

23. The method according to claim 1,
wherein the donor cells (6) are donor cells of non-human mammals.

24. (canceled)

25. (canceled)

26. (canceled)
